(19) United States
(12) Patent Application Publication
(10) Pub. No.: US 2025/0179418 A1
(43) Pub. Date: Jun. 5, 2025

(54) UNIVERSAL MICROBIAL SAMPLE TRANSPORT MEDIUM FOR GENOMIC AND METAGENOMIC STUDIES

(71) Applicant: TRANSLATIONAL HEALTH SCIENCE AND TECHNOLOGY INSTITUTE, Faridabad (IN)

(72) Inventors: Bhabatosh Das, Faridabad (IN); Shruti Panwar, Faridabad (IN); Nitya Wadhwa, Faridabad (IN); Roshan Kumar, Faridabad (IN)

(73) Assignee: TRANSLATIONAL HEALTH SCIENCE AND TECHNOLOGY INSTITUTE, Faridabad (IN)

(21) Appl. No.: 18/834,547

(22) PCT Filed: Apr. 14, 2023
(86) PCT No.: PCT/IB2023/053829

§ 371 Date: Jul. 30, 2024

(30) Foreign Application Priority Data
Apr. 20, 2022 (IN) 202211023269

(51) Int. Cl. C12N 1/04

(52) U.S. Cl. CPC C12N 1/04; C12N 2500/30; C12N 2500/35

(57) ABSTRACT

The present invention is drawn to a novel buffer composition comprising a nuclease quenching agent, a stabilizer, a salt, and a cryoprotectant. The present invention is also drawn to a process of producing the composition of the present invention and its utility. The composition of the present invention supports viability of aerobic and anaerobic microbes for extended periods over significant range of temperatures, without the presence of any significant amount of degradants of the constituents of the samples such as biological macromolecules and DNA. The present invention also relates to an economically viable and wide range applications for storage of biological and environmental samples containing microbes.

### FIELD OF THE INVENTION

The present invention relates to the field of biotechnology and microbiology. In particular, the present invention relates to development of an aqueous buffer composition for collection, transport, and storage of a biological specimen containing a population of microbes.

### BACKGROUND OF THE INVENTION

The need of optimal buffers in biological and clinical researches is universal. However, in the past, very few buffers are available in the important pH range. Those that were available were not economical and compatible for biological research and had serious disadvantages, such as toxicity or undesired reactivity. Phosphate buffers, for example, exhibit poor buffering capacity above pH 7.5, and they often inhibit reactions and precipitate polyvalent cations. Buffers below pH 7.5 such as TRIS can be toxic and show poor buffering capacity. Similarly, glycylglycine is useful above pH 8, but is of no value below pH 7.5. There are several important criteria that must be met in order for a buffer to be useful in biological systems. The buffers must be enzymatically and hydrolytically stable. The pKa of the buffer should be between 6 and 8 for most biological reactions. The pH of the buffer solution should be minimally affected by concentration, temperature, ionic composition, or salt effects of the medium. The buffer should be soluble in water and relatively insoluble in other solvents. The buffer should have soluble cationic complexes. The buffer should exhibit no absorption of light in either the visible or UV regions. Further, the buffer needs to be amenable for maintaining the structural integrity and stability for the macromolecules, during temperature fluctuations that may be incurred during transportation.

DNA preservation buffers should have constituents that inhibits the degradation of DNA molecules. Chemicals such as salts, silica beads, Ethylenediaminetetraacetic acid (EDTA), ethanol, Dimethyl sulfoxide (DMSO) etc. reduce microbial growth and inactivate proteins with enzymatic activity. Certain kits are available commercially such as DNA Genotek kit, DNAgard, NorgenBiotek and RNA later which allows the entire protocol with ease. However, most of these kits are very expensive. In addition, such buffers don't support the viability of aerobic and anaerobic microbes. One of the buffers used by certain research groups for sample transportation is the Nucleic acid preservation (NAP) buffer. NAP buffer was initially propounded by Miguel Camacho-Sanchez and colleagues. However, the composition of this buffer involves a huge amount of Ammonium Sulphate salts. It uses acids like Sulphuric acid to lower the pH. Use of acids in buffers leads to issues of microbial viability and integrity, and that of acid susceptible biological macromolecules. In addition, NAP buffer is not suitable for supporting microbial viability in the freezing condition. Hence, there is an urgent need to develop a buffer that would be economic and suitable for preserving nucleic acids and support the viability of aerobic and anaerobic microbes in the biospecimen.

### OBJECT OF THE INVENTION

It is an object of the present invention to overcome the drawbacks of the prior art. It is another object of the present invention to provide a novel buffer composition for preservation of nucleic acids.

It is another object of the present invention to provide a novel buffer composition to support the viability of aerobic and anaerobic microbes in a biospecimen.

### SUMMARY OF THE INVENTION

Accordingly, the present invention provides a novel buffer composition comprising a nuclease quenching agent, a stabilizer, a salt, and a cryoprotectant.

According to another aspect of the present invention there is provided a process for preparing the buffer composition, said process comprising the steps of:
i. dissolving said salt, said nuclease quenching agent and said stabilizer in water to obtain a solution;
ii. adding said cryoprotectant to the solution of step (i) to obtain a uniform solution and rendering the solution to a desired volume;
iii. autoclaving said solution of step (ii) at 121° C. for 15 minutes to obtain said buffer composition.

### DETAILED DESCRIPTION OF THE INVENTION

The present invention provides a novel collection, transport and storage buffer composition comprising a nuclease quenching agent, a stabilizer, a salt, and a cryoprotectant.

The composition of the present invention includes a nuclease quenching agent. The nuclease quenching agent may be selected from pyrophosphoric acid, citric acid, tripolyphosphoric acid, EDTA, EGTA and Glycine. More preferably, the nuclease quenching agent of the present invention is EDTA. The nuclease quenching agent may be present in a range of 10 mM-30 mM of the composition; and most preferably 20-25 mM of the composition of the present invention.

The composition of the present invention includes a stabilizer. The stabilizer may be selected from Sodium salt, citric acid, potassium citrate, sodium citrate and potassium phosphate. More preferably, the stabilizer of the present invention is a sodium citrate. The stabilizer may be present in a range of 10 mM-30 mM of the composition; and most preferably 20-26 mM of the composition of the present invention.

The composition of the present invention includes a salt. The composition of the present invention comprises 26% reduction of salts in comparison to other prior art compositions for the same purpose. The salt may be selected from sodium sulphate, potassium sulphate, ammonium sulphate and ammonium acetate. More preferably, the salt of the present invention is sodium sulphate and ammonium sulphate. Most preferably, the salt is ammonium sulphate. The salt may be present in a range of 3M-5M of the composition; and most preferably 3.7M of the composition of the present invention.

The salt may maintain the pH range of the composition, in a range of 7.0-7.5, preferably 7.3 to 7.5. The composition of the present invention maintains the 80%-100% microbes alive for 0-180 days, when stored at a temperature range of −80° C.-85° C.

The composition of the present invention includes a cryoprotectant. The cryoprotectant may be selected from sugar, polyol, polymer, glycerol and amino acid. More preferably, the cryoprotectant of the present invention is a polyol. Most preferably, the cryoprotectant is glycerol. The cryoprotectant may be present in a range of 5%-10% of the composition; and most preferably 2-6% of the composition of the present invention.

The present invention also provides a process of producing the composition of the present invention and its utility. The buffer solution in accordance with the present invention is prepared by adding salts, stabilizers, nuclease quenching agents, and cryoprotectant in a specific order at an optimum temperature. Throughout in the preparation phase, temperature was maintained from 40° C.-50° C. For dissolving all the components, a magnetic stirrer (300 rpm) was used in microbe free environment. Finally, an autoclave was used to sterilize the buffer solution at 121° C. for 15 minutes.

The composition of the present invention supports viability of aerobic and anaerobic microbes (bacteria and fungi) for extended periods over significant range of temperatures, without the presence of any significant amount of degradants of the constituents of the samples such as biological macromolecules and DNA. The present invention also relates to an economically viable and wide range applications for storage of biological and environmental samples containing microbes.

In an embodiment, there is provides a process for preparing the composition of the present invention, said process comprising the steps of:
i dissolving salt, nuclease quenching agent and the stabilizer in water to obtain a solution;
ii addition of cryoprotectant to the solution of step (i) to obtain a uniform solution and rendering the solution to a desired volume;
iii autoclaving the solution of step (ii) at 121° C. for 15 minutes to obtain the composition of the present invention.

Not bound by any theory it has been found that the order of the addition of salts, temperature of the environment and aeration is critical in achieving the composition of the present invention.

The dissolution of salt, nuclease quenching agent and the stabilizer in water in step (i) generally takes place at about 45° C.

The aqueous medium of the present invention may preferably be sterile, and may contain resistivity in the range of 10 to 25 MΩ, more preferably in the range of 15 to 20 MΩ. It has been found that an excess metal ion contamination in the aqueous medium may reduce the efficiency of nuclease quenching agent and affects nucleic acid integrity over time.

The composition of the present invention can be distributed in suitable aliquots depending on commercial requirement.

In another aspect of the invention is to provide a buffer composition that allows the maintenance of viability of microbes and the integrity of nucleic acids in clinical samples at different temperatures.

Without being limited by theory, it is proposed that the optimum use of the various ingredients at the said ratio maintains the stability of the composition during storage and use. The composition of the present invention is also synergistic in that the ingredients when constituted together as per the principles herein yield a composition having appropriate activity and stability during its shelf-life.

### ADVANTAGES OF THE COMPOSITION OF THE PRESENT INVENTION

The composition of the present invention allows the maintenance of viability of microbes and the integrity of nucleic acids in clinical samples at different temperatures.
The composition of the present invention results in a good yield of nucleic acids from the clinical samples. It works at different temperatures including room temperature ranging from 25° C. to −85° C., hence allowing the easy transport of the precious samples and allows hassle free storage. The composition of the present invention maintains the quality and quantity of macromolecules intact for certain duration mostly at freezing condition.

Further the composition of the present invention maintains the viability of microbes and preserve the integrity of nucleic acids for long time at wide range of temperatures like 4° C., −20° C. and −80° C.

The present invention is illustrated by examples. The examples are mere embodiments of the present invention and cannot be construed as limiting.

### Example 1: Composition of the Present Invention

The composition of present invention, a novel stable storage buffer consists of a nuclease quenching agent, a stabilizer, a salt, and a cryoprotectant. The composition of the present invention includes a nuclease quenching agent; most preferably the nuclease quenching agent is EDTA (20-30 mM). The composition of the present invention includes a stabiliser; most preferably the stabiliser is sodium citrate (20-30 mM). The present invention consists of a salt, most preferably the salt is Ammonium sulphate (3-5M). It also contains a cryoprotective agent, and most preferably the cryoprotective agent is Glycerol (5-10%). A few illustrative compositions of the present invention are provided at Table 1.2 and 3.

TABLE 1
[Composition for the present invention]

| S.No. | Chemicals | Concentration | Importance |
|---|---|---|---|
| 1. | EDTA | 25.4 mM | Quencher of nuclease stabilising ions |
| 2. | Sodium | 24.99 mM | Stabilizes the DNA and protect its |
| | citrate | | integrity in the fecal sample |
| 3. | Ammonium | 3.7M | Hygroscopic in nature and |
| | sulphate | | important for DNA integrity |
| 4. | Glycerol | 5% | Works as a cryoprotectant |

TABLE 2
[Composition for the present invention]

| | Composition | Composition | Composition | Composition |
|---|---|---|---|---|
| Chemical | 1 | 2 | 3 | 4 |
| EDTA | 25.4 mM | 25.4 mM | 25.4 mM | 25.4 mM |
| Sodium | 24.99 mM | 24.99 mM | 24.99 mM | 24.99 mM |
| citrate | | | | |
| Ammonium | 5M | 5M | 3.7M | 3.7M |
| sulphate | | | | |
| Glycerol | 5% | Absent | Absent | 5% |

A few illustrative compositions are shown in Table 1 and 2.

TABLE 3
[Composition for the present invention]

| | Composition | Composition | Composition | Composition |
|---|---|---|---|---|
| | 1 | 2 | 3 | 4 |
| Solubility | Solution | Solution | Excellent | Excellent |
| | supersaturated | supersaturated | | |
| | Salt | Salt | | |
| | Precipitated | Precipitated | | |
| pH | Slightly | Slightly | Neutral | Neutral |
| | acidic (5.1) | acidic (5.1) | (7.4) | (7.4) |
| Microbial | Excellent | Decreased | Decreased | Excellent |
| viability & DNA | | | | |
| Integrity | | | | |
| Downstream | Robust | Not Appropriate | Not Appropriate | Robust |
| processes | | | | |
| (PCR, DNA | | | | |
| Sequencing, | | | | |
| Restriction | | | | |
| Digestion) | | | | |

The compositions as tried in Tables 1 and 2 of the present invention was tested for various parameters such as solubility, pH, microbial viability & DNA Integrity and processes such as PCR, DNA Sequencing, Restriction Digestion by standard assays, and the results are presented at Table 3.

From the Table 3, it can be seen that when the components are present as per the composition of the present invention, the composition possesses desired properties and efficacy as ascribed in the specification. The components of the composition act in a synergistic manner to provide the requisite properties and efficacy.

### Example 2: Process for Preparing the Composition of the Present Invention

The process for preparation of the composition of the present invention includes combining 1.86 g of EDTA, 1.84 g of sodium citrate trisodium salt dihydrate, and 122.2 g of ammonium sulphate in 150 ml of water in bottle or flask. Stirring the composition on low to moderate heat until the ammonium sulphate dissolves completely. Once dissolved adding 12.5 ml of glycerol. Making up the volume to 250 ml. Storing at room temperature or keeping refrigerated until aliquoted. Autoclaving the solution before usage and then taking aliquot 1.5 mL of buffer into 2 mL tubes for preservation of up to 150 mg of sliced tissue.

### Example 3: Process for Storage of Fecal Sample in Sample Transport Medium

The process of collecting, transporting and storing the fecal sample in microbial sample transport medium, includes

Using 150-220 mg fresh or freeze stored stool sample. Transferring sample into pre-chilled microcentrifuge tube (MCT). Resuspending sample in 200 ul TE buffer (50 mM Tris-1 mM EDTA, Ph8.0). Adding 4 glass beads (2.5 mm) in the MCT and vortex continuously for 1 min. or until the sample was thoroughly homogenized. Adding enzyme cocktail containing 50 ul Lysozyme (10 mg/ml), 6 ul mutanolysin (25 KU/ml), and 3 ul lysostaphin (4 KU/ml) and incubating for 1 hour at 37° C. Adding 250 ul Guanidine thiocyanate (4M) and mixing gently for 45 seconds. Adding 120 ul 1N-Lauryl sarcosine (10%) and waiting for 10 min. Adding 1500 ul 1 N-Lauryl sarcosine (5%). Turning on the heating block at 70° C. Vortexing to make a homogenous solution. Short spinning the samples at 14000 rpm for few seconds. Incubating at 70° C. in the heating block for 1-2 hours. Transferring the sample into bead beating tubes (approx. 800 ul per tube). Adding 750 ul bead-beater beads (0.1 mm) and cutting off the outermost part of the pipette tip. Avoiding adding the top fraction of water in the tip. Homogenizing on the bead-beater with 15 mg PVPP NB in 30 sec beating and 30 sec wait pattern. Vortexing until PVPP is well mixed. Spinning at 14000 rpm for 3 minutes. Transferring the supernatant to a new 5 ml tube (approx. 800 ul). Keeping both the supernatant and the pellet. Washing the pellet with 500 ul TENP and vortexing. Spinning at 14000 rpm for 3 minutes and pooling the supernatants. Washing the pellet with 500 ul TENP and vortexing. Spinning at 14000 rpm for 3 minutes and pooling the supernatants. Spinning the pooled supernatant at 14000 rpm. Transferring the supernatant to two new 5 ml tubes of each sample. Adding 2 ml isopropanol to each tube and mixing by inverting the tubes a few times. Incubating for 10 minutes at Room temperature. Spinning at 14000 rpm for 10 minutes. Discarding as much as supernatant possible. Drying the pellet in the incubator at 42° C. for 1 hour, possibly longer if pellet is not dry. Dissolving the pellet by adding 900 ul phosphate buffer and 100 ul potassium acetate and pipette up and down until the pellet gets dissolved. NB generates foam and is time consuming. Incubating on ice for a minimum of 90 minutes or store at −20° C. overnight. Turning on the heating block at 37° C. and making the centrifuge cool down to 4° C. Defrosting the samples until there is no longer ice in them and spinning at 14000 rpm for 30 min. at 4° C. Transferring the supernatant from both tubes to a new 5 ml tube. Adding 4 ul RNAse (10 mg/ml), mixing and spinning down shortly. Incubating for 1 hour at 37° C. Adding 100 ul sodium-acetate 3M. Adding 2 ml ice cold 96% ethanol. Inverting the tubes few times to mix. Incubating for 15 min. at Room Temperature. Spinning at 14000 rpm for 15 min. at 4° C. discarding the ethanol with a pipette. Washing the pellet with 1 ml 75% ethanol and vortexing the sample. Spinning at 14000 rpm for 15 min. at 4° C. discard the ethanol with a pipette. Drying the pellet in the incubator at 42° C. for 1 hour. Resuspending the pellet in 200 ul TE buffer pH 8. Incubating at Room temperature for 10-15 min at 4° C. Resuspending the DNA by pipetting up and down. Spinning at 14000 rpm for 10 minutes. Transferring supernatant to a new 2 ml tube. Storing samples at −80° C.

### Results

Successful isolation of large amount (>1 μg) of quality DNA from all the tested samples was achieved using the universal microbial sample transport medium. The total yield of DNA was much higher as compared to yield of DNA through kits.

The universal microbial sample transport medium maintained the Integrity of DNA sample and microbial viability in all the samples.

TABLE 4
Storage of Fecal Sample in
Sample Transport Medium]

| | | Date of | Date of | |
|---|---|---|---|---|
| | Sample | sample | sample | Type of |
| S.No. | ID | receiving | processing | sample |
| 1. | S418641 | Dec. 23, 2020 | Dec. 24, 2020 | Fecal |
| 2. | 2279 | Jan. 5, 2021 | Jan. 6, 2021 | Fecal |
| 3. | 418763 | Jan. 8, 2021 | Jan. 9, 2021 | Fecal |
| 4. | 418756 | Jan. 8, 2020 | Jan. 9, 2020 | Fecal |
| 5. | 1816 | Jan. 9, 2020 | Jan. 10, 2020 | Fecal |
| 6. | 2647 | Jan. 14, 2021 | Jan. 15, 2021 | Fecal |
| 7. | 2750 | Jan. 23, 2021 | Jan. 24, 2021 | Fecal |
| 8. | 2555 | Feb. 2, 2021 | Feb. 3, 2021 | Fecal |
| 9. | 2556 | Feb. 3, 2021 | Feb. 4, 2021 | Fecal |
| 10. | 2650 | Feb. 4, 2021 | Feb. 5, 2021 | Fecal |
| 11. | 1696 | Feb. 7, 2021 | Feb. 8, 2021 | Fecal |
| 12. | S111824 | Feb. 12, 2021 | Feb. 13, 2021 | Fecal |
| 13. | 1697 | Feb. 13, 2021 | Feb. 14, 2021 | Fecal |
| 14. | 2752 | Feb. 14, 2021 | Feb. 15, 2021 | Fecal |
| 15. | S111899 | Feb. 14, 2021 | Feb. 16, 2021 | Fecal |
| 16. | S84735 | Feb. 25, 2021 | Feb. 26, 2021 | Fecal |
| 17. | 2755 | Feb. 27, 2021 | Feb. 28, 2021 | Fecal |
| 18. | S84805 | Mar. 10, 2021 | Mar. 11, 2021 | Fecal |
| 19. | S84820 | Mar. 12, 2021 | Mar. 13, 2021 | Fecal |
| 20. | 2660 | Mar. 27, 2021 | Mar. 28, 2021 | Rectal |
| | | | | swab(RC) |
| 21. | 2764 | Apr. 7, 2021 | Apr. 8, 2021 | Fecal |
| 22. | 2662 | Apr. 8, 2021 | Apr. 9, 2021 | Fecal |
| 23. | 2487 | Apr. 8, 2021 | Apr. 9, 2021 | Fecal |
| 24. | S84992 | Apr. 8, 2021 | Apr. 9, 2021 | Fecal |
| 25. | S85002 | Apr. 9, 2021 | Apr. 10, 2021 | Fecal |
| 26. | S112223 | Apr. 12, 2021 | Apr. 13, 2021 | RC |
| 27. | 1703 | Feb. 14, 2021 | Feb. 15, 2021 | RC |
| 28. | 1703 | Feb. 14, 2021 | Feb. 15, 2021 | Fecal |
| 29. | 2765 | Apr. 15, 2021 | Apr. 16, 2021 | RC |
| 30. | 2766 | Apr. 16, 2021 | | RC |
| 31. | 2814 | Apr. 17, 2021 | May 11, 2021 | Fecal |
| 32. | 2768 | Apr. 20, 2021 | May 11, 2021 | Fecal |
| 33. | 1704 | Apr. 24, 2021 | | RC |
| 34. | 1704 | Apr. 25, 2021 | May 11, 2021 | Fecal |
| 35. | 2705 | Apr. 28, 2021 | May 11, 2021 | Fecal |
| 36. | 1818 | Apr. 29, 2021 | May 11, 2021 | Fecal |
| 37. | 2663 | May 5, 2021 | May 11, 2021 | Fecal |
| 38. | 2769 | May 16, 2021 | May 20, 2021 | Fecal |
| 39. | 2770 | May 17, 2021 | May 20, 2021 | Fecal |
| 40. | 2707 | May 19, 2021 | May 20, 2021 | Fecal |
| 41. | 2771 | May 26, 2021 | Jun. 14, 2021 | Fecal |
| 42. | 2709 | May 28, 2021 | Jun. 14, 2021 | Fecal |
| 43. | 2708 | May 28, 2021 | Jun. 14, 2021 | Fecal |
| 44. | 1820 | Jun. 4, 2021 | Jun. 14, 2021 | Fecal |
| 45. | 2711 | Jun. 5, 2021 | Jun. 14, 2021 | Fecal |
| 46. | 2710 | Jun. 8, 2021 | Jun. 24, 2021 | Fecal |
| 47. | 1821 | Jun. 9, 2021 | Jun. 24, 2021 | Fecal |
| 48. | 2772 | Jun. 10, 2021 | Jun. 24, 2021 | Fecal |
| 49. | 2713 | Jun. 10, 2021 | Jun. 24, 2021 | Fecal |
| 50. | 2712 | Jun. 11, 2021 | Jun. 24, 2021 | Fecal |
| 51. | 2714 | Jun. 15, 2021 | Jun. 24, 2021 | Fecal |
| 52. | 2773 | Jun. 17, 2021 | Jun. 24, 2021 | Fecal |
| 53. | 1822 | Jun. 18, 2021 | Jun. 24, 2021 | Fecal |
| 54. | 2774 | Jun. 19, 2021 | Jun. 24, 2021 | Fecal |

### Examples Showing Transport and Storage in the Buffer:

Transport and storage of meconium and fecal samples: The components of the buffer are not a mere admixture but are synergistic in nature. It helps to maintain the DNA integrity and viability of aerobic and anaerobic bacterial species. In the absence of cryoprotective agent, viability of microbes in freezing condition is substantially reduced. Similarly, in the absence of nuclease quenching agent the integrity of the nucleic acid also get compromised within weeks of time.
Transport and storage of environmental swab samples: The components of the buffer help to maintain the DNA integrity and viability of aerobic and anaerobic bacterial species. The present inventors have tested viability of Candida spp. and observed that they are viable in this solution even after 180 days of storage at −80° C.

### Examples in Leading to the Buffer of the Present Invention:

The components of the buffer prevent any sharing of genomic DNA, from the fecal samples collected and stored in the buffer. The DNA from the fecal samples stored in the buffer were isolated. The isolated DNA comigrate with 23-Kbp DNA fragments in a 0.8% agarose gel, indicated that a good quality of DNA was recovered.

Examples Showing that the Components of the Buffer are not a Mere Admixture but are Synergistic in Nature:

It has been found that both aerobic and anaerobic bacterial species stay viable in the present buffer for a longer period of time (>90 days) Reduction of CFU numbers were not observed in the fecal sample after storage in the deep freezer with the present buffer.

The reported sample transport buffer of the prior art was designed to maintain DNA integrity. The buffer composition of the present invention not only maintains DNA integrity but also Cell viability.

TABLE 5
Synergistic Effect of the components in the buffer composition of the present invention

| | | Buffer | Buffer | Buffer | Buffer |
|---|---|---|---|---|---|
| | | Composition | Composition | Composition | Composition |
| Characteristics | | 1 | 2 | 3 | 4 |
| Microbial | E. coli | 2.0 × 10⁹/ml | 2.7 × 10⁷/ml | 2.3 × 10⁷/ml | 2.3 × 10¹⁰/ml |
| viability | Candida | 6.0 × 10⁸/ml | 4.3 × 10⁷/ml | 2.4 × 10⁷/ml | 3.1 × 10⁹/ml |
| DNA | E. coli | 19 μg | 13 μg | 11 μg | 21 μg |
| Integrity/ | Candida | 7.0 μg | 5.0 μg | 6.0 μg | 9 μg |
| yield | | | | | |

The buffer composition 1, 2, 3 and 4 are mentioned in Table 2

To establish the synergistic effect of different compounds used in developing the current buffer, the different compositions of the present invention, as mentioned in Table 5, were tested for bacterial and fungal viability and also for the integrity of their genomic DNA. From Table 5, it is evident that when the components are present as per the composition of the present invention, the bio-specimen preserved in freezing condition maintains the best bacterial and fungal viability and yields the highest amount of genomic DNA. Here, the viability of the microbes was tested using two model organisms E. coli and Candida sp. commonly used in laboratories for multiple purposes, including diagnostic testing and drug discovery. The components of the present composition act in a synergistic manner to provide the requisite properties and efficacy.

1. A buffer composition comprising a nuclease quenching agent, a stabilizer, a salt, and a cryoprotectant.

2. The buffer composition as claimed in claim 1, wherein said nuclease quenching agent is selected from pyrophosphoric acid, citric acid, tripolyphosphoric acid, EDTA, EGTA and Glycine.

3. The buffer composition as claimed in claim 1, wherein said nuclease quenching agent is EDTA.

4. The buffer composition as claimed in claim 1, wherein said nuclease quenching agent is present in a range of 10 mM-30 mM of the composition.

5. The buffer composition as claimed in claim 1, wherein said stabilizer is selected from sodium salt, citric acid, potassium citrate, sodium citrate and potassium phosphate.

6. The buffer composition as claimed in claim 1, wherein said stabilizer is sodium citrate.

7. The buffer composition as claimed in claim 1, wherein said stabilizer is present in a range of 10 mM-30 mM of the composition.

8. The buffer composition as claimed in claim 1, wherein said salt is selected from sodium sulphate, potassium sulphate, ammonium sulphate and ammonium acetate.

9. The buffer composition as claimed in claim 1, wherein said salt is selected from sodium sulphate and ammonium sulphate.

10. The buffer composition as claimed in claim 1, wherein said salt is present in a range of 3M-5M of the composition.

11. The buffer composition as claimed in claim 1, wherein said cryoprotectant is selected from sugar, polyol, polymer, glycerol and amino acid.

12. The buffer composition as claimed in claim 1, wherein said cryoprotectant is a polyol.

13. The buffer composition as claimed in claim 1, wherein said cryoprotectant is present in a range of 5%-10% of the composition.

14. The buffer composition as claimed in claim 1, wherein said composition has a pH in a range of 7.0 to 7.5.

15. The buffer composition as claimed in claim 1, wherein said composition provides economically viable and wide range applications for storage of biological and environmental samples containing microbes.

16. The buffer composition as claimed in claim 1, wherein said composition maintains 80%-100% microbes alive for 0-180 days, when stored at a temperature range of −80° C.-85° C.

17. The buffer composition as claimed in claim 1, wherein said composition has a resistivity in the range of 10 to 25 MΩ.

18. A process for preparing the buffer composition as claimed in claim 1, said process comprising the steps of:
i) dissolving said salt, said nuclease quenching agent and said stabilizer in water to obtain a solution;
ii) adding said cryoprotectant to the solution of step (i) to obtain a uniform solution and rendering the solution to a desired volume; and
iii) autoclaving said solution of step (ii) at 121° C. for 15 minutes to obtain said buffer composition.

19. The process as claimed in claim 18, wherein said dissolving in step (i) is performed at 45° C.
